(19) United States
(12) Patent Application Publication
(10) Pub. No.: US 2021/0038799 A1
(43) Pub. Date: Feb. 11, 2021

(54) FACILITATING CONNECTION OF HOME MEDICAL DEVICE TUBING

(71) Applicant: Fresenius Medical Care Holdings, Inc., Waltham, MA (US)

(72) Inventors: Eric Bergman, Newton, MA (US); Jessica Steuber, Ashland, MA (US); David Yuds, Hudson, NH (US); Jon F. Moss, Antioch, CA (US); Jonathan Leclerc, Northborough, MA (US); Maria Tamayo-Coffey, Pleasanton, CA (US); Samiullah K. Durrani, Harvard, MA (US); Rachel Bartels, Somerville, MA (US)

(21) Appl. No.: 16/533,086

(22) Filed: Aug. 6, 2019

(51) Int. Cl. A61M 1/28; A61M 1/16; A61M 39/10

(52) U.S. Cl. CPC A61M 1/287; A61M 1/1656; A61M 39/10; A61M 1/282; A61M 2205/128; A61M 2205/50; A61M 2205/33; A61M 1/281

(57) ABSTRACT

A tubing connection system is disclosed by which patients or others seeking to make connections between tubing sets, particularly for a home dialysis machine, can make those connections without having to hold each tube in each hand. In various implementations, the tubing connection device may include mechanisms for rectilinear and rotational motion to engage tubing connectors for connection between the tubing connectors as well as torque control features to prevent over-tightening. The system may also include automatically controlled gripping mechanisms to facilitate gripping of the tubing in connection with tubing connection operations. The system provides for facilitating a consistently secure connection of the tubing connectors.

### TECHNICAL FIELD

This application relates generally to systems and methods for facilitating contact-minimized (or hands-free) connection of medical device tubing, particularly tubing used with home dialysis machines.

### BACKGROUND

Medical devices such as dialysis machines are known for use in the treatment of renal disease. The two principal dialysis methods are hemodialysis (HD) and peritoneal dialysis (PD). During hemodialysis, the patient's blood is passed through a dialyzer of a hemodialysis machine while also passing dialysate through the dialyzer. A semi-permeable membrane in the dialyzer separates the blood from the dialysate within the dialyzer and allows diffusion and osmosis exchanges to take place between the dialysate and the blood stream. During peritoneal dialysis, the patient's peritoneal cavity is periodically infused with dialysate, or dialysis solution. The membranous lining of the patient's peritoneum acts as a natural semi-permeable membrane that allows diffusion and osmosis exchanges to take place between the solution and the blood stream. Automated peritoneal dialysis machines, also called PD cyclers, are designed to control the entire peritoneal dialysis process so that it can be performed at home, usually overnight, without clinical staff in attendance. Both HD and PD machines may include displays with touch screens or other user interfaces that display information of a dialysis treatment and/or enable an operator or patient to interact with the machine.

Dialysis machines may have a disposable set which has several connectors and tubing, also referred to herein as “lines,” used in connection with the dialysis treatment and through which medical fluid flows during the dialysis treatment. Home dialysis patients are required to make multiple tubing connections in the process or setting up their home dialysis machine. These tubing connections are usually made with a Luer Lock connector, having male and female compatible components, and involves twisting the tubing a quarter turn to make a clean and secure connection between tubing paths, for example, to and from disposable sets, solution bags, the patient, and a drain line.

It is important that the connectors not touch dirty surfaces and become contaminated. One of the most common medical risks faced by PD patients is infection in the form of peritonitis. Peritonitis typically occurs due to a “failure” of aseptic technique when a surface of a tubing connector handled by the patient comes in contact with a non-sterile surface. Such surfaces may include the patient's hands (if not properly cleaned), the patient's clothing, or various household objects.

A related challenge for some patients is that it can be difficult to connect tubes without increased risk of failure of aseptic technique. Underlying causes of these challenges include the fact that some patients may have tremors due to comorbidities, such as Parkinson's disease or incipient tremor, or that some patients may be handicapped with impaired use of one hand (e.g., due to arthritis or stroke) or no use of one hand (e.g., due to stroke, amputation etc.). Still other patients have issues with peripheral neuropathy leading to numbness or pain in their extremities. All of these issues can impact effectiveness of aseptic technique, and they can also lead to connections that are not entirely snug, with potential for leaks. For example, Luer Lock type connections that require twisting may be a difficult maneuver for patients with dexterity issues. This is because one hand must tightly grip on side of the tubing connection while the other hand must tightly grip the other side of the tubbing connection and make a one-quarter turn twisting motion. If the connector is not twisted enough or if the tubing on the connector is twisted such that it untwists its own connector, it will leak fluid out or leak air into the system, exposing the patient to spill hazards or air embolism. If the tubing is over-torqued, disconnecting the tubing may become a source of frustration.

One existing solution by which challenges in facilitating and maintaining aseptic technique has been addressed is the stay-safe® organizer, a device developed and distributed by Fresenius Medical Care. The organizer permits a PD user to place one end of a tubing connector into a holder thus enabling them to make connections using only one hand while one side of the tubing is held stationary in the organizer. That said, patients who have tremor and other issues are still subject to potential unintentional touching of connector surfaces because they still must use a hand to unscrew and attach the connectors, and when they move the tubing segments together with the intent of connecting them, at this point there is risk of unintentional touch contamination. It is also noted that organizer is designed for use with particular types of connection components.

Accordingly, it would be desirable to provide a system that addresses the issues noted above, including issues of inadvertent failure of aseptic technique via touch contamination and the challenges faced by patients/users who have mobility impairments impacting effective use of one or more hands.

### SUMMARY

According to the system described herein, a tubing connection device comprises a first tubing holder that receives a first tubing connector and a second tubing holder that is movable and receives a second tubing connector that is connectable to the first tubing connector. A drive mechanism drives movement of the second tubing holder toward the first tubing holder and drives a motion of the second tubing holder that enables connection of the first tubing connector with the second tubing connector after contact of the first tubing connector and the second tubing connector. The drive mechanism further includes a torque controller that controls an amount of torque that is exerted between the first tubing connector and the second tubing connector when the first tubing connector and the second tubing connector are in contact with each other and engaged in a connection operation. In various implementations, the drive mechanism may include a mechanically actuated drive mechanism and/or a motor driven drive mechanism. The second tubing holder may be driven in a rotation motion to enable connection of the first tubing connector and the second tubing connector. The first tubing holder may include an automatic grip device that automatically grips the first tubing connector. The automatic grip device may include a motor, a processor and at least one sensor, and data from the at least one sensor may be used by the processor to control, via the motor, a gripping action of the automatic grip device of the first tubing connector. The sensor may include one or more of a touch sensor, an optical sensor and/or a voice-actuated sensor. The first tubing connector and the second tubing connector may be compatible Luer Lock connectors.

According further to the system described herein, a dialysis tubing connection system comprises a first tubing set having a first tubing connector, a disposable tubing set that is couplable to a dialysis machine and that includes a second tubing connector, and a tubing connection device. The tubing connection device comprises a first tubing holder that receives a first tubing connector and a second tubing holder that is movable and receives a second tubing connector that is connectable to the first tubing connector. A drive mechanism drives movement of the second tubing holder toward the first tubing holder and drives a motion of the second tubing holder that enables connection of the first tubing connector with the second tubing connector after contact of the first tubing connector and the second tubing connector. The drive mechanism further includes a torque controller that controls an amount of torque that is exerted between the first tubing connector and the second tubing connector when the first tubing connector and the second tubing connector are in contact with each other and engaged in a connection operation. In various implementations, the drive mechanism may include a mechanically actuated drive mechanism and/or a motor driven drive mechanism. The second tubing holder may be driven in a rotation motion to enable connection of the first tubing connector and the second tubing connector. The first tubing holder may include an automatic grip device that automatically grips the first tubing connector. The automatic grip device may include a motor, a processor and at least one sensor, and data from the at least one sensor may be used by the processor to control, via the motor, a gripping action of the automatic grip device of the first tubing connector. The sensor may include one or more of a touch sensor, an optical sensor and/or a voice-actuated sensor. The first tubing connector and the second tubing connector may be compatible Luer Lock connectors.

According further to the system described herein, a method for facilitating tubing connections comprises positioning a first tubing connector in a first tubing holder and positioning a second tubing connector in a second tubing holder that is movable. The method further comprises driving, via a driving mechanism, movement of the second tubing holder toward the first tubing holder, and driving, via the driving mechanism, a motion of the second tubing holder that enables connection of the first tubing connector with the second tubing connector after contact of the first tubing connector and the second tubing connector. The method further comprises controlling, via a torque controller, an amount of torque that is exerted between the first tubing connector and the second tubing connector when the first tubing connector and the second tubing connector are in contact with each other and engaged in a connection operation. The second tubing connector may be a connector at an end of a disposable tubing set that is couplable to a dialysis machine.

### BRIEF DESCRIPTION OF THE DRAWINGS

Embodiments and features of the system described herein are explained with reference to the several figures of the drawings, which are briefly described as follows. FIG. 1 illustrates an exemplary embodiment of a dialysis machine, specifically a PD machine, in a dialysis system configured in accordance with the system described herein.

FIG. 2 is a perspective view of the PD machine and the PD cassette of the PD system of FIG. 1.

FIG. 3 is a schematic illustration showing tubing paths among the patient, the PD machine and a drain when the patient is receiving a PD treatment in accordance with the system described herein.

FIG. 4 is a schematic illustration showing a hands-free line connector tool according to an embodiment of the system described herein.

FIG. 5 is a schematic illustration showing connection of a bag line connector of tubing from a dialysate bag to a disposable line connector on tubing of a disposable set from the PD machine.

FIG. 6 is a schematic illustration showing another implementation of a line connector tool that may include an electric motor driven mechanism activated by a press of a button.

FIG. 7 is a schematic illustration showing another embodiment of a tubing connection system implemented as an automatic grip device.

FIG. 8 is a schematic illustration showing a connection operation by a patient using the automatic grip device.

FIG. 9 is a schematic illustration showing an automatic line connection tool including use of an automatic grip device with a drive mechanism according to an embodiment of the system described herein.

FIG. 10 is a flow diagram showing processing for facilitating a tubing connection according to an implementation of the system described herein.

### DETAILED DESCRIPTION

The system described herein provides for a device by which patients or others seeking to make connections between tubing sets, particularly for a home medical device, can make those connections without having to hold each tube in each hand or, in some implementations, without having to touch any tube during the time of attachment. The system provides for facilitating a consistent secure connection of the tubing connectors.

FIG. 1 shows an example of a medical device, implemented as a peritoneal dialysis (PD) system 100, that is configured in accordance with an exemplary embodiment of the system described herein. In some implementations, the PD system 100 may be configured for use at a patient's home (e.g., a home dialysis system). The PD system 100 may include a dialysis machine 102, e.g. a PD machine, also referred to as a PD cycler, and in some embodiments may be seated on a cart 104. The dialysis machine 102 may include a housing 106, a door 108, and a cartridge interface for contacting a disposable PD cassette, or cartridge, when the cartridge is disposed within a compartment formed between the cartridge interface and the closed door 108. A heater tray 116 may be positioned on top of the housing 106. The heater tray 116 may be any size and shape to accommodate a bag of dialysate (e.g., a 5 L bag of dialysate). The dialysis machine 102 may also include a user interface such as a touch screen 118 and control panel 120 operable by a user (e.g., a caregiver or a patient) to allow, for example, set up, initiation, and/or termination of a PD treatment.

Dialysate bags 122 may be suspended from the sides of the cart 104, and a heater bag 124 may be positioned in the heater tray 116. Hanging the dialysate bags 122 may improve air management as any air is disposed by gravity to a top portion of the dialysate bag 122. Valves may be attached to a bottom portion of the dialysate bags 122 so fluid is drawn out and air delivery is minimized. Dialysate from the dialysate bags 122 may be transferred to the heater bag 124 in batches. For example, a batch of dialysate may be transferred from the dialysate bags 122 to the heater bag 124, where the dialysate is heated by the heating element. When the batch of dialysate has reached a predetermined temperature (e.g., approximately 98°-100° F., 37° C.), the batch of dialysate may be flowed into the patient. The dialysate bags 122 and the heater bag 124 may be connected to the cartridge via dialysate bag lines 126 and a heater bag line 128, respectively. As illustrated, for example, each bag line, such as a dialysate bag line 126, may include a disposable tubing and connector set 126a coupled to a bag tubing and connector set 126b. The dialysate bag lines 126 may be used to pass dialysate from dialysate bags 122 to the cartridge during use, and the heater bag line 128 may be used to pass dialysate back and forth between the cartridge and the heater bag 124 during use. In addition, a patient line 130 and a drain line 132 may be connected to the cartridge. The patient line 130 may be connected to a patient's abdomen via a catheter and may be used to pass dialysate back and forth between the cartridge and the patient's peritoneal cavity during use. The drain line 132 may be connected to a drain or drain receptacle and may be used to pass dialysate from the cartridge to the drain or drain receptacle during use.

The touch screen 118 and the control panel 120 may allow a user to input various treatment parameters to the dialysis machine 102 and to otherwise control the dialysis machine 102. In addition, the touch screen 118 may serve as a display. The touch screen 118 may function to provide information to the patient and the operator of the PD system 100. For example, the touch screen 118 may display information related to a dialysis treatment to be applied to the patient, including information related to a prescription. In various embodiments, the control panel 120 may also include audio and video component capabilities, including speakers, microphones and/or cameras.

The dialysis machine 102 may include a processing module 101 that resides inside the dialysis machine 102, the processing module 101 being configured to communicate with the touch screen 118 and the control panel 120. The processing module 101 may be configured to receive data from the touch screen 118 the control panel 120 and sensors, e.g., temperature and pressure sensors, and control the dialysis machine 102 based on the received data. For example, the processing module 101 may adjust the operating parameters of the dialysis machine 102.

The dialysis machine 102 may be configured to connect to a network 110. The connection to network 110 may be via a wireless connection, or in some cases a wired connection, as further discussed elsewhere herein. The dialysis machine 102 may include a connection component 111 configured to facilitate the connection to the network 110 along with the processing module 101. The connection component 111 may be a transceiver for wireless connections and/or other signal processor for processing signals transmitted and received over a wired connection. In the case of a wired connection, the connection component 111 may be a port enabling a physical connection to a network component, such as a network modem. Other medical devices (e.g., other dialysis machines) or components may be configured to connect to the network 110 and communicate with the dialysis machine 102.

Although the present disclosure is discussed herein principally in connection with a peritoneal dialysis machine, the system described herein may be used and implemented in connection with other configurations of PD machines, as well as other types of medical devices, including home hemodialysis machines, that require tubing connections.

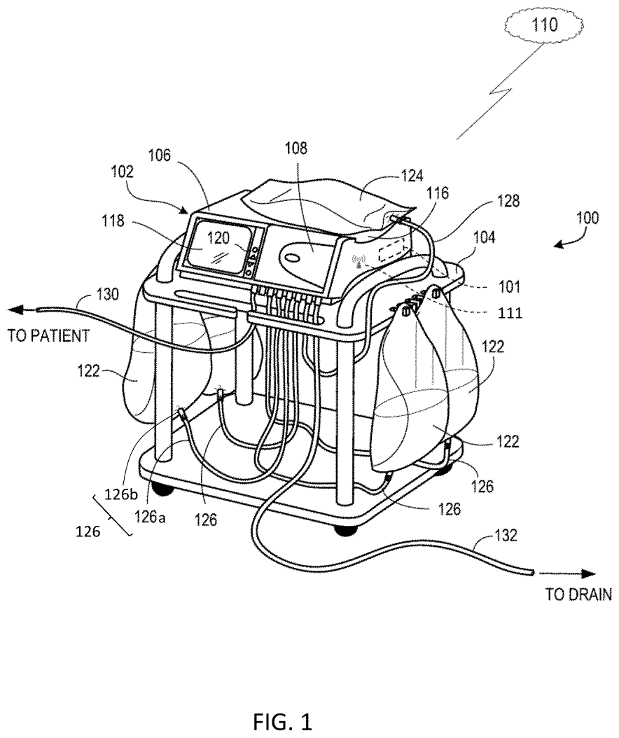

FIG. 2 is a perspective view of the PD machine 102 and the PD cassette 112 of the PD system 100 of FIG. 1, in accordance with some embodiments. As depicted in FIG. 2, the PD cassette 112 is placed proximate the cassette interface 109. The cassette 112 contains pump chambers 138A, 138B, pressure sensing chambers 163A, 163B, and valve chambers for controlling the flow of fluid through the cavities of the cassette 112. The cassette 112 is connected to the dialysate bag lines 126, the heater bag line 128, the patient line 130, and the drain line 132, including via connection of disposable set lines 126a of the cassette 112 having connectors that may be used to connect the disposable set lines of the cassette 112 to connectors of lines from the dialysate bags, heater bag, patient lines etc., including via use of a line organizer.

The cassette interface 109 includes a surface having holes formed therein. The PD machine 102 includes pistons 133A, 133B with piston heads 134A, 134B attached to piston shafts (not explicitly shown). The piston shafts can be actuated to move the piston heads 133A, 133B axially within piston access ports 136A, 136B formed in the cassette interface 109. The pistons 133A, 133B are sometimes referred to herein as pumps. In some embodiments, the piston shafts can be connected to stepper motors that can be operated to move the pistons 133A, 133B axially inward and outward such that the piston heads 134A, 134B move axially inward and outward within the piston access ports 136A, 136B. The stepper motors drive lead screws, which move nuts inward and outward on the lead screws. The stepper motors can be controlled by driver modules. The nuts, in turn, are connected to the piston shafts, which cause the piston heads 134A, 134B to move axially inward and outward as the stepper motors drive the lead screws. Stepper motor controllers provide the necessary current to be driven through the windings of the stepper motors to move the pistons 133A, 133B. The polarity of the current determines whether the pistons 133A, 133B are advanced or retracted. In some embodiments, to the stepper motors require 200 steps to make a full rotation, and this corresponds to 0.048 inches of linear travel of the piston heads 134A, 134B.

In some embodiments, the PD system 100 also includes encoders (e.g., optical quadrature encoders) that measure the rotational movement and direction of the lead screws. The axial positions of the pistons 133A, 133B can be determined based on the rotational movement of the lead screws, as indicated by feedback signals from the encoders. Thus, measurements of the position calculated based on the feedback signals can be used to track the position of the piston heads 134A, 134B of the pistons 133A, 133B.

When the cassette 112 is positioned within the cassette compartment 114 of the PD machine 102 with the door 108 closed, the piston heads 134A, 134B of the PD machine 102 align with the pump chambers 138A, 138B of the cassette 112 such that the piston heads 134A, 134B can be mechanically connected to dome-shaped fastening members of the cassette 112 overlying the pump chambers 138A, 138B. As a result of this arrangement, movement of the piston heads 134A, 134B toward the cassette 112 during treatment can decrease the volume of the pump chambers 138A, 138B and force dialysate out of the pump chambers 138A, 138B. Retraction of the piston heads 134A, 134B away from the cassette 112 can increase the volume of the pump chambers 138A, 138B and cause dialysate to be drawn into the pump chambers 138A, 138B.

The cassette 112 also includes pressure sensor chambers 163A, 163B. When the cassette 112 is positioned within the cassette compartment 114 of the PD machine 102 with the door 108 closed, pressure sensors 151A, 151B align with the pressure sensor chambers 163A, 163B. Portions of a membrane that overlies the pressure sensor chambers 163A, 163B adhere to the pressure sensors 151A, 151B using vacuum pressure. Specifically, clearance around the pressure sensors 151A, 151B communicates vacuum to the portions of the cassette membrane overlying the pressure sensing chambers 163A, 163B to hold those portions of the cassette membrane tightly against the pressure sensors 151A, 151B. The pressure of fluid within the pressure sensing chambers 163A, 163B causes the portions of the cassette membrane overlying the pressure sensor chambers 163A, 163B to contact and apply a force to the pressure sensors 151A, 151B. The pressure sensors 151A, 151B can be any sensors that are capable of measuring the fluid pressure in the pressure sensor chambers 163A, 163B. In some embodiments, the pressure sensors are solid state silicon diaphragm infusion pump force/pressure transducers.

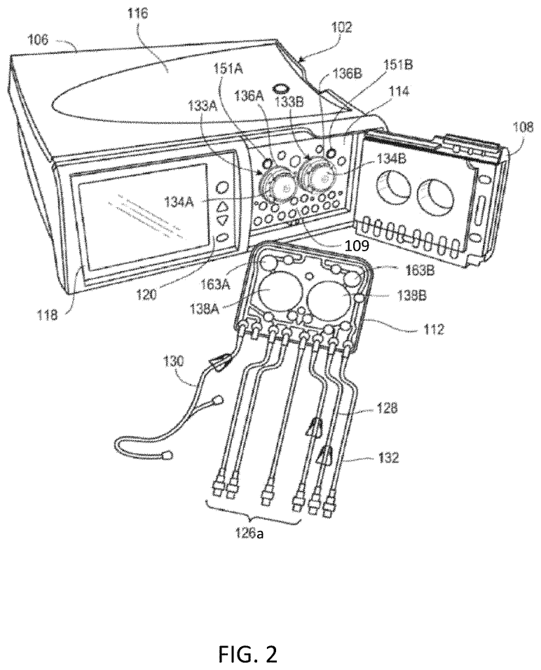

FIG. 3 is a schematic illustration showing tubing paths among the patient, the PD machine 102, a drain when the patient is receiving a PD treatment in accordance with the present disclosure. A proximal end of the patient line 130 is connected to the cassette 112 that is installed in the PD machine 102. A distal end of the patient line 130 is connected to the patient's abdomen 176 via a catheter 172. The catheter 172 is connected to the patient line via a port 174. A proximal end of the drain line 132 is connected to the cassette 112, and a distal end of the drain line 132 is connected to a drain 180 or a drain receptacle such as a bag, tub, or other receptacle capable of holding fluid. In some embodiments, the lines may be connected via an intervening line organizer, such as the stay-safe® organizer made by Fresenius Medical Care. It will be appreciated that the distal end of the drain line 132 can be open to the air in order to promote fluid discharge into the drain 180. In connection with setting up the PD machine 102 in the home, the patient or caregiver is responsible for appropriately connecting the lines to and from the PD machine 102, including the patient line 130 and the drain line 132, as well as the bag lines 126, 128 discussed elsewhere herein.

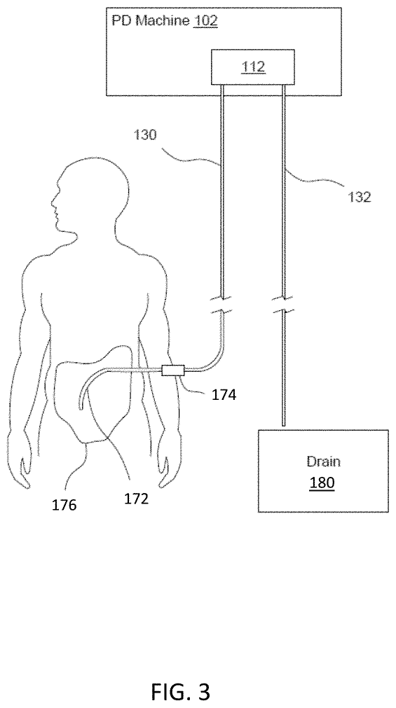

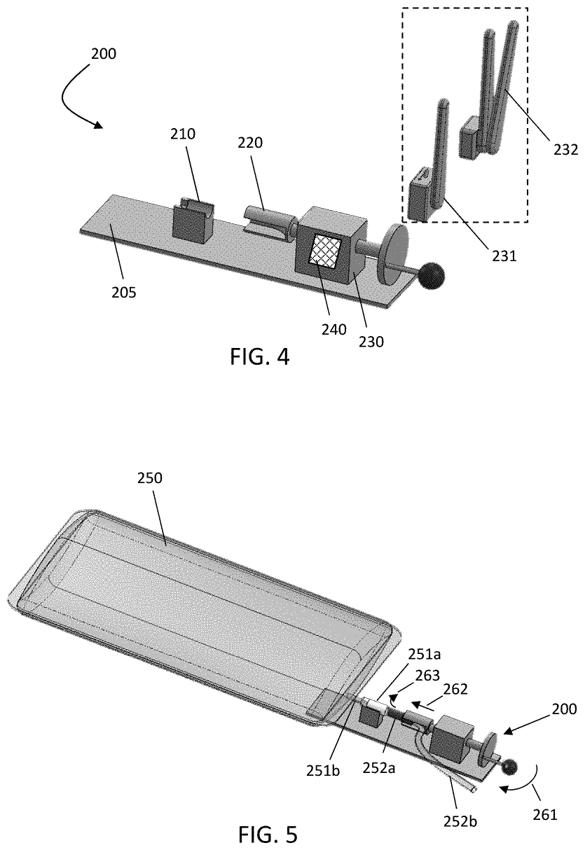

FIGS. 4 and 5 show schematic illustrations of a hands-free line connector tool 200 and its operation for connecting a dialysate bag line according to an embodiment of the system described herein. FIG. 4 shows components of the contact-minimized (hands-free) line connector tool 200 according to one or more implementations, and FIG. 5 is a schematic illustration showing connection of a bag line connector 251a of tubing 251b from a dialysate bag 250 to a disposable line connector 252a on tubing 252b of a disposable set from the PD machine 102 (e.g. forming one of the lines 126). The hands-free line connector tool 200 provides for minimized contact of patients/users with tubing by eliminating the need for patients/users to touch the tubing after the connector caps are removed, eliminating the need for patients/users to handle tubing, and/or eliminating the need for patient/users to align the tubing while making the connection. As discussed in detail below, the system described herein allows the user to place both sides of the tubing in holders of the hands-free line connector tool 200 and then a mechanism drives the tubing segments together creating a secure connection. The system described herein may be used for any tubing connection utilizing described connectors.

In an implementation, the line connector tool 200 may include a first line holder 210, e.g. the bag line holder for holding a line connector from the dialysate bag, a second line holder 220, e.g., a disposable line holder for holding a line connector from a disposable set of the PD machine 102 to be connected to the dialysate bag, and a drive mechanism 230 for driving connection of the tubes in the holders. As illustrated, the drive mechanism 230 may be implemented as mechanical actuation mechanism, such as a rotating crank mechanism using, for example, a threaded screw rod configuration and rotational crank. In other implementations involving mechanical actuation, which are illustrated as alternatives in the figure in the dashed-line box, other types of drive mechanisms may include a ratchet mechanism 231 and/or a squeeze action mechanism 232 having a grip handle that drives gearing inside the body of the mechanism 230, and which may also include spring-loading components for reversal. The line connector tool 200 may include a base 205 that remains stationary when the line connector tool 200 is in use, which in various implementations may be achieved by sufficient weight to not move significantly or by clamping or adhering (e.g. suction cups, hook-and-loop fasteners, etc.) to a surface.

The connector 251a that is on the tubing 251b from the bag 250 is held in place in the first (bag) line holder 210 and cannot rotate or move. It can be placed using only one hand. The connector 252a on the disposable set tubing 252b from the cycler disposable set may be positioned on the second (disposable set) line holder 220. The positioning of the connectors 251a, 252a on the line connector tool 200 may enable the user to remove the connector caps from each of the lines, but uncapped surfaces of the connectors subject to aseptic technique should not touch the line connector tool 200. After placement of the connectors 251a, 252a, the drive mechanism 230 is engaged. For example, the drive mechanism 230 may be engaged by circular motion (e.g. arrow 261) of the rotating crank mechanism to cause rectilinear motion (e.g. arrow 262) of the line holder 220 towards the bag line holder 210 to a point where the connectors 251a, 252a make contact. As discussed herein, other implementations of the drive mechanism 230 and concomitant motion techniques are possible and contemplated.

The disposable line holder 220 has the ability to rotate but only after engagement with the connector in the bag line holder 210. That is, after the two connectors 251, 252a make full contact, the drive mechanism 230 provides for the disposable line holder 220 to rotate (e.g. arrow 263) the connector 252a clockwise until a “thumb tight” connection is established. In an embodiment, the thumb tight connection may be established by a torque controller 240, e.g. torque limited gearing of the drive mechanism 230 and/or other appropriate torque limiter or disconnect mechanisms, e.g. gearsets, friction plates, magnetic mechanisms etc., at which point the rotational movement of the disposable set line holder 220 is ended. The disposable set line holder 220 may then be disengaged from the connector 252a, leaving the user free to pull the now connected lines from the bag line holder 210. This connection process may be reversible, in that a user may have the disposable set line holder 220 fully extended, then put the connectors into place in each holder and be able to disconnect the lines by moving the disposable set line holder 220 in reverse, causing it to counter rotate to unscrew the connection to the bag 250.

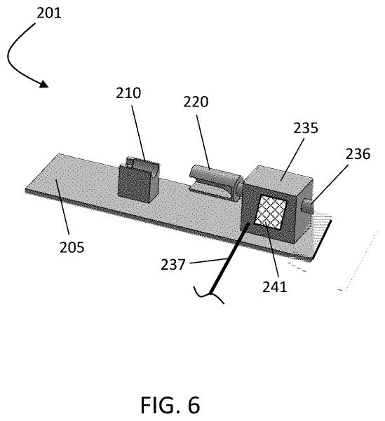

FIG. 6 is a schematic illustration showing another implementation of a line connector tool 201 that may include an electric motor driven mechanism 235 activated by a press of a button 236 or similar actuation interface. The electric motor driven mechanism 235 may further include a power connection 237 to provide power to the electric motor component of the mechanism 235 and which may include a connection to the dialysis machine 102 and/or to an external power source. By pressing the button 236, the electric motor driven mechanism 235 may automatically drive the disposable set line holder 220 to the bag line holder 210 to further minimize necessary action of the user to make the tubing connection. Other components and operations of the line connector tool 201 may be similar to those elsewhere described herein with respect to the line connector tool 200. The line connector tool 201 may also include a torque controller 241, having components similar to those described above in connection with the torque controller 240 and/or may further include other motor torque control features, e.g. torque control based on control of current to the motor driven mechanism 235 etc.

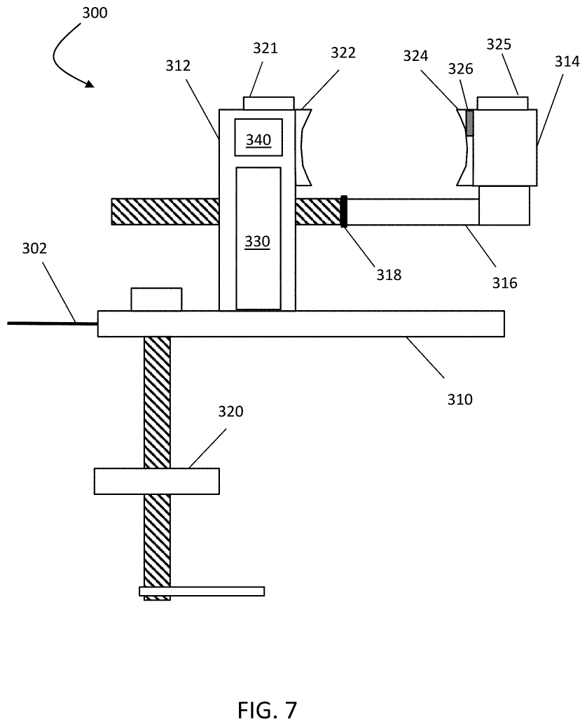

FIG. 7 is a schematic illustration showing another embodiment of a tubing connection system implemented as an automatic grip device 300. The automatic grip device 300 may be operated with one hand and may include sensors and safeguards to prevent too tight of a grip on the tubing connection, no matter the type of tubing connection, and allow for patients with dexterity issues to simply place the desired lines into the device 300 and safely connect or disconnect the lines with one hand. The device 300 may resemble a vise and include a base 310 and a mechanism 320 for securing the device 300 to a tabletop, such as a clamping mechanism. Further, the device 300 includes a fixed grip jaw 312 and a movable grip jaw 314 that is adjustable (e.g., extend or retract) via a threaded arm 316. The threaded arm 316 may include a physical stop 318 to prevent over tightening of the grip on inserted tubing. Each of the grip jaws 312, 314 may include sensors 322, 324, such as touch or optical sensors, that enable the device 300 to detect the tubing connector placed in the jaws 312, 314 and then automatically and gently secure the connector in place via use of a motor 330 that drives extension and retraction of the threaded arm 316 and motion of the moveable jaw 314. Other sensors 321, 325, such as touch or voice-actuated sensors, may be used to control opening and closing of the jaw 314. Processing of input from the sensors used to control the gripping action provided by motion of the moveable jaw 314, as well as other other processing, may be performed by an onboard processor 340 of the device 300. The device 300 may also include other sensors, such as an infrared sensor 326, to prevent pinching of the operator. A power connection 302 may be provided to power the device 300 and which may be connected to the dialysis machine 102 and/or to an external power source.

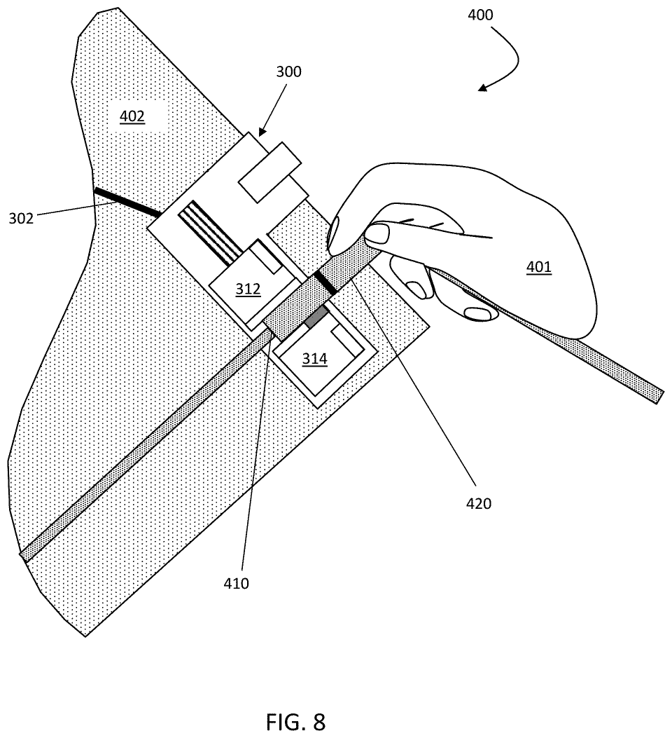

FIG. 8 is a schematic illustration showing a connection operation 400 by a patient 401 using the automatic grip device 300. The device 300 may be secured to a tabletop 402. To allow one-handed operation to open or close automatically the device 300, the device 300 may rely on the sensors 321-326 such that the user may hold the tubing, for example the bag line connector 410, in a designated location for a period of time, e.g. 2 seconds, and/or the user may issue a voice command and the device 300 would gently close the grip and tighten appropriately when the user has vacated his or her and from the area. The patient 401 may then connect the disposable set line connector 420 to the bag line connector 410, that is held in place by the device 300, by the standard Luer Lock connection quarter turn. Thereafter, another gesture or touch or voice prompt would cause the device 300 to open again, for example, once the tubing is held by the user to prevent it from being dropped.

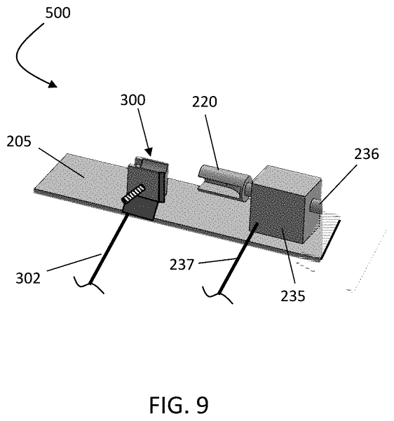

FIG. 9 is a schematic illustration showing an automatic line connection tool 500 including use of the automatic grip device 300 along with components of a hands-free line connection tool, for example the hands-free line connection tool 201 like that discussed above, according to an embodiment of the system described herein. As illustrated, the automatic line connection tool 500 may include use of the automatic grip device 300 used as the bag line holder, e.g. replacing the bag line holder 210 in the line connection tool 201 having the components as discussed above. It is noted that the automatic line connection tool 500 may also be implemented in connection with other versions of line connection tools, including the manual operated line connection tool 200 and alternative drive mechanisms thereof.

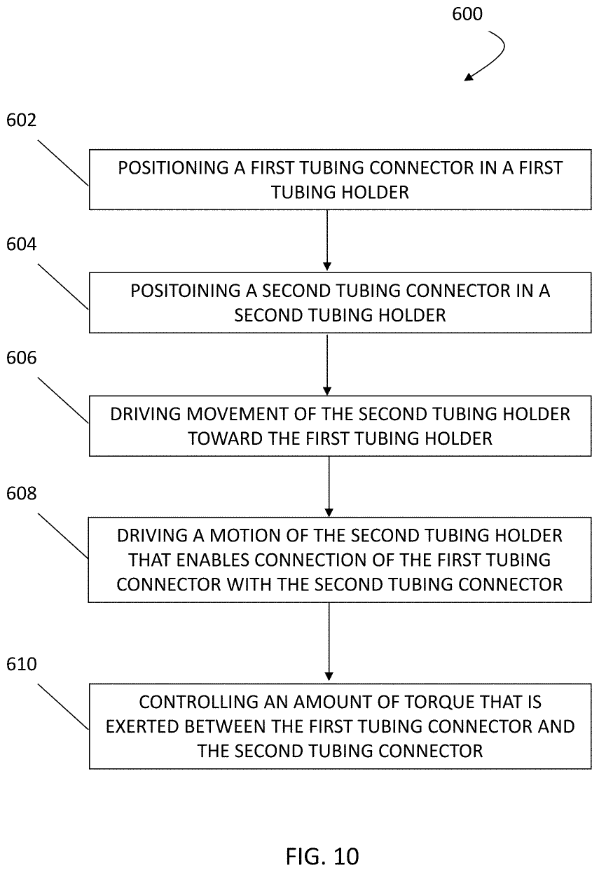

FIG. 10 is a flow diagram 600 showing processing for facilitating a tubing connection according to an implementation of the system described herein. At a step 602, a first tubing connector is positioned in a first tubing holder. It is noted that the positioning may be manually positioning in a static tubing holder and/or automatically positioned via an automatic grip device like that discussed elsewhere herein. At a step 604, a second tubing connector is positioned in a second tubing holder that is movable. At a step 606, movement of the second tubing holder is driven, via a driving mechanism, toward the first tubing holder. At a step 608, a motion of the second tubing holder is driven, via the driving mechanism, that enables connection of the first tubing connector with the second tubing connector after contact of the first tubing connector and the second tubing connector. At a step 610, an amount of torque that is exerted between the first tubing connector and the second tubing connector is controlled, via a torque controller, such as a torque limiter, when the first tubing connector and the second tubing connector are in contact with each other and engaged in a connection operation.

Implementations discussed herein may be combined with each other in appropriate combinations in connection with the system described herein. Additionally, in some instances, the order of steps in the flow diagrams, flowcharts and/or described flow processing may be modified, where appropriate. The system may further include a display and/or other computer components for providing a suitable interface with a user and/or with other computers. Aspects of the system described herein may be implemented or controlled using software, hardware, a combination of software and hardware and/or other computer-implemented or computer-controlled modules or devices having described features and performing described functions. Data exchange and/or signal transmissions to, from and between components of the system may be performed using wired or wireless communication. This communication may include use of one or more transmitter or receiver components that securely exchange information via a network, such as the Internet, and may include use of components of local area networks (LANs) or other smaller scale networks, such as Wi-Fi, Bluetooth or other short range transmission protocols, and/or components of wide area networks (WANs) or other larger scale networks, such as mobile telecommunication networks.

Software implementations of aspects of the system described herein may include executable code that is stored in a computer-readable medium and executed by one or more processors. The computer-readable medium may include volatile memory and/or non-volatile memory, and may include, for example, a computer hard drive, ROM, RAM, flash memory, portable computer storage media, an SD card, a flash drive or other drive with, for example, a universal serial bus (USB) interface, and/or any other appropriate tangible or non-transitory computer-readable medium or computer memory on which executable code may be stored and executed by a processor. The system described herein may be used in connection with any appropriate operating system. The meanings of any method steps of the invention(s) described herein are intended to include any suitable method of causing one or more parties or entities to perform the steps unless a different meaning is expressly provided or otherwise clear from the context.

As used herein, an element or operation recited in the singular and preceded with the word “a” or “an” should be understood as not excluding plural elements or operations, unless such exclusion is explicitly recited. References to “one” embodiment or implementation of the present disclosure are not intended to be interpreted as excluding the existence of additional embodiments that also incorporate the recited features. Furthermore, a description or recitation in the general form of “at least one of [a], [b] or [c],” or equivalent thereof, should be generally construed to include [a] alone, [b] alone, [c] alone, or any combination of [a], [b] and [c].

Implementations of the invention will be apparent to those skilled in the art from a consideration of the specification or practice of the invention disclosed herein. It is intended that the specification and examples be considered as exemplary only, with the true scope and spirit of the invention being indicated by the following claims.

What is claimed is:

1. A tubing connection device, comprising:
a first tubing holder that receives a first tubing connector;
a second tubing holder that is movable and receives a second tubing connector that is connectable to the first tubing connector;
a drive mechanism that drives movement of the second tubing holder toward the first tubing holder and drives a motion of the second tubing holder that enables connection of the first tubing connector with the second tubing connector after contact of the first tubing connector and the second tubing connector, wherein the drive mechanism further includes a torque controller that controls an amount of torque that is exerted between the first tubing connector and the second tubing connector when the first tubing connector and the second tubing connector are in contact with each other and engaged in a connection operation.

2. The tubing connection device according to claim 1, wherein the drive mechanism includes a mechanically actuated drive mechanism.

3. The tubing connection device according to claim 1, wherein the drive mechanism includes a motor driven drive mechanism.

4. The tubing connection device according to claim 1, wherein the second tubing holder is driven in a rotation motion to enable connection of the first tubing connector and the second tubing connector.

5. The tubing connection device according to claim 1, wherein the first tubing holder includes an automatic grip device that automatically grips the first tubing connector.

6. The tubing connection device according to claim 5, wherein the automatic grip device includes a motor, a processor and at least one sensor, and wherein data from the at least one sensor is used by the processor to control, via the motor, a gripping action of the automatic grip device of the first tubing connector.

7. The tubing connection device according to claim 6, wherein the at least one sensor includes one or more of a touch sensor, an optical sensor or a voice-actuated sensor.

8. The tubing connection device according to claim 1, wherein the first tubing connector and the second tubing connector are compatible Luer Lock connectors.

9. A dialysis tubing connection system, comprising:
a first tubing set having a first tubing connector;
a disposable tubing set that is couplable to a dialysis machine, wherein the disposable set includes a second tubing connector; and
a tubing connection device, the tubing connection device comprising:
a first tubing holder that receives a first tubing connector;
a second tubing holder that is movable and receives a second tubing connector that is connectable to the first tubing connector;
a drive mechanism that drives movement of the second tubing holder toward the first tubing holder and drives a motion of the second tubing holder that enables connection of the first tubing connector with the second tubing connector after contact of the first tubing connector and the second tubing connector, wherein the drive mechanism further includes a torque controller that controls an amount of torque that is exerted between the first tubing connector and the second tubing connector when the first tubing connector and the second tubing connector are in contact with each other and engaged in a connection operation.

10. The dialysis tubing connection system according to claim 9, wherein the disposable tubing set is a tubing set for a peritoneal dialysis machine.

11. The dialysis tubing connection system according to claim 9, wherein the disposable tubing set is a tubing set for a home hemodialysis machine.

12. The dialysis tubing connection system according to claim 9, wherein the drive mechanism includes a mechanically actuated drive mechanism.

13. The dialysis tubing connection system according to claim 9, wherein the drive mechanism includes a motor driven drive mechanism.

14. The dialysis tubing connection system according to claim 9, wherein the second tubing holder is driven in a rotation motion to enable connection of the first tubing connector and the second tubing connector.

15. The dialysis tubing connection system according to claim 9, wherein the first tubing holder includes an automatic grip device that automatically grips the first tubing connector.

16. The dialysis tubing connection system according to claim 15, wherein the automatic grip device includes a motor, a processor and at least one sensor, and wherein data from the at least one sensor is used by the processor to control, via the motor, a gripping action of the automatic grip device of the first tubing connector.

17. The dialysis tubing connection system according to claim 16, wherein the at least one sensor includes one or more of a touch sensor, an optical sensor or a voice-actuated sensor.

18. The dialysis tubing connection system according to claim 9, wherein the first tubing connector and the second tubing connector are compatible Luer Lock connectors.

19. A method for facilitating tubing connections, comprising:
positioning a first tubing connector in a first tubing holder;
positioning a second tubing connector in a second tubing holder that is movable;
driving, via a driving mechanism, movement of the second tubing holder toward the first tubing holder;
driving, via the driving mechanism, a motion of the second tubing holder that enables connection of the first tubing connector with the second tubing connector after contact of the first tubing connector and the second tubing connector;
controlling, via a torque controller, an amount of torque that is exerted between the first tubing connector and the second tubing connector when the first tubing connector and the second tubing connector are in contact with each other and engaged in a connection operation.

20. The method according to claim 19, wherein the second tubing connector is a connector at an end of a disposable tubing set that is couplable to a dialysis machine.
